(19) United States
(12) Patent Application Publication
(10) Pub. No.: US 2025/0222100 A1
(43) Pub. Date: Jul. 10, 2025

(54) COMPLETE TOLEROGENIC ADJUVANT AND METHODS OF USING THE SAME

(71) Applicant: THE BOARD OF TRUSTEES OF THE UNIVERSITY OF ILLINOIS, URBANA, IL (US)

(72) Inventors: GUOXING ZHENG, ROCKFORD, IL (US); AOSHUANG CHEN, ROCKFORD, IL (US)

(73) Assignee: THE BOARD OF TRUSTEES OF THE UNIVERSITY OF ILLINOIS, URBANA, IL (US)

(21) Appl. No.: 19/008,835

(22) Filed: Jan. 3, 2025

Related U.S. Application Data
(60) Provisional application No. 63/617,553, filed on Jan. 4, 2024.

(51) Int. Cl. A61K 39/39; A61K 31/436; A61K 31/573; A61K 39/00; A61K 39/35; A61P 37/06

(52) U.S. Cl. CPC A61K 39/39; A61K 31/436; A61K 31/573; A61K 39/0008; A61K 39/35; A61P 37/06; A61K 2039/55511; A61K 2039/55572

(57) ABSTRACT

Provided herein is a complete tolerogenic adjuvant composition comprising an effective amount of dexamethasone, an effective amount of rapamycin, and an effective amount of a toll-like receptor agonist and method of using the same to induce regulatory T cell-mediated antigen-specific immunosuppression and treat or reduce the risk of developing an autoimmune disease or condition, allergy, transplant rejection or graft versus host disease.

### CROSS-REFERENCE TO RELATED APPLICATIONS

This application claims benefit from U.S. Provisional Patent Application Ser. Nos. 63/617,553, filed Jan. 4, 2024, the contents of which are incorporated herein by reference in their entireties.

### FEDERALLY SPONSORED RESEARCH AND DEVELOPMENT

This invention was made with government support under grant no. R21 HL106340 awarded by the National Institutes of Health. The government has certain rights in this invention.

### BACKGROUND OF THE INVENTION

Autoimmunity, allergy, and graft rejection are all perpetuated by an adaptive immune response that is both antigen-specific and already active. Inducing an antigen-specific immunosuppression against this kind of response may provide for a cure of these immune diseases.

One approach to t goal is to raise endogenous immunosuppressive cells, particularly CD4⁺CD25⁺Foxp3⁺ regulatory T (“Treg”) cells specific for an antigen that is causing the adaptive immune (“recall antigen”). To that end, a different kind of vaccine adjuvant called “tolerogenic adjuvant” that permits raising the Treg cells via immunization has been suggested (Kang et al. (2008) J. Immunol. 180:5172-5176). Immunization is arguably the simplest way of raising T cells. But to raise the Treg cells selectively, a novel adjuvant is needed to prevent other T cells, many of which are pathogenic and already active during disease, from reacting to immunization.

Because of that need, tolerogenic adjuvants have been sought mostly from “biased” immunosuppressants that inhibit the conventional types of CD4⁺ T (“Teff”) cells while sparing Treg cells. The synthetic glucocorticoid dexamethasone was the first tolerogenic adjuvant (Kang et al. (2008) J. Immunol. 180:5172-5176), chosen for this bias (Chen et al. (2004) Eur. J. Immunol. 34:859-869; Chen et al. (2006) Eur. J. Immunol. 36:2139-2149). In mouse models, immunization with a recall antigen and dexamethasone led to preferential expansion of recall antigen-specific Treg cells (over Teff cells) that correlated with suppression of established allo- and autoimmunity (Kang et al. (2008) J. Immunol. 180:5172-5176). The term “suppressed immunization” was coined for this form of immunization that combines antigen and immunosuppressant (Kang et al. (2008) J. Immunol. 180:5172-5176). Subsequently, various other immunosuppressants, including immunomodulators, were explored (Northrup et al. (2017). Mol. Pharm. 14:66-80; Northrup et al. (2016) Adv. Drug Deliv. Rev. 98:86-98). Collectively, these studies showed the generality of using biased immunosuppressants as tolerogenic adjuvants. The combination of antigen and immunosuppressant is now a common part of many forms of antigen-specific immunotherapy besides suppressed immunization.

Mechanistic understanding of how biased immunosuppressants work as tolerogenic adjuvants has come mostly from studies of dexamethasone (Stagliano & Oppenheim (2013) Eur. J. Immunol. 43:38-41). As an analog of the body's naturally produced immunosuppressant, dexamethasone preferentially inhibits not only Teff cells, but also other cell types that are essential for a Teff response, in a pattern that shows coordination. In mice, dexamethasone kills Teff cells, B-2 cells, and dendritic cells (“DCs”) while sparing Treg cells, B-1 cells, and the CD11c^{lo}CD40^{lo} monocyte-derived macrophages (“MMφs”; Chen et al. (2004) Eur. J. Immunol. 34:859-869; Chen et al. (2006) Eur. J. Immunol. 36:2139-2149; Chen et al. (2014) J. Immunol. 193:35-39; Zheng et al. (2013) Eur. J. Immunol. 43:219-227). Dexamethasone kills cells by engaging glucocorticoid receptor-α (“GR-α”), whereas cells can avoid being killed by expressing glucocorticoid receptor-β (“GR-β”), a dominant-negative isoform (Hinds et al. (2010) Mol. Endocrinol. 24:1715-1727; Oakley & Cidlowski (2011) J. Biol. Chem. 286:3177-3184). Therefore, the GR isoform hypothesis has been proposed, wherein the pattern of bias seen among the aforementioned cell types is set intrinsically via the expression of the GR isoforms (Zheng et al. (2013) Eur. J. Immunol. 43:219-227). Influenced by this pattern, Treg cells, B-1 cells, and MMos are enriched at the expense of Teff cells, B-2 cells, and DCs during suppressed immunization (Chen et al. (2014) J. Immunol. 193:35-39; Zheng et al. (2013) Eur. J. Immunol. 43:219-227). Among the enriched cells, MMØS differentiate into tolerogenic antigen presenting cells that activate Treg cells but not memory Teff cells (Zheng et al. (2013) Eur. J. Immunol. 43:219-227). These studies thus point to MMos as a mediator for tolerogenic adjuvants.

Despite these advances, there is a pressing need to improve tolerogenic adjuvants. Biased immunosuppressants are not true adjuvants by the conventional standard because they lack the ability to expand, via immunization, antigen-specific Treg cells to a greater absolute number than that via immunization with antigen alone. Rather, by relying on inhibiting Teff cells, they expand Treg cells only relatively (versus the Teff cells), which limits efficacy. Preventing Teff cells from reacting to immunization is not the only condition a tolerogenic adjuvant must meet. To be more efficacious, the adjuvant should also have the ability to expand Treg cells in absolute numbers, thereby positively stimulating a Treg response. Not meeting this second condition, most conventional, biased immunosuppressants are actually “incomplete” adjuvants.

### SUMMARY OF THE INVENTION

This disclosure provides a complete tolerogenic adjuvant composition and vaccine comprising an effective amount of dexamethasone, an effective amount of rapamycin, and an effective amount of a Toll-like receptor agonist that increases influx of monocyte-derived macrophages into lymph nodes (e.g., a TLR7/8 agonist such as resiquimod or a TLR4 agonist such as monophosphoryl lipid A), e.g., in a mass ratio of about 8:20:3 or about 8:20:6, and methods of using the same to induce regulatory T cell-mediated antigen-specific immunosuppression and/or treat or reduce the risk of developing an autoimmune disease or condition, allergy, transplant rejection or graft versus host disease.

### BRIEF DESCRIPTION OF DRAWINGS

FIG. 1 shows that rapamycin (“Rapa”) blocks monophosphoryl lipid A (“MPLA”)-induced influx of Teff cells to DLN. Shown are dose-response curves generated by nonlinear regression. Left: absolute cell counts for MMos, DCs, Treg cells, and Teff cells one day after a single injection into a footpad of a graded dose of MPLA. Right: the same set of cells after injection of 3 μg (based on the left panel) of MPLA mixed with a graded dose of Rapa. Each dose point is mean±SD of four experiments. * p≤0.03 and ** p≤0.01, for Teff and MMφ counts, respectively, at MPLA doses of ≥1 μg versus 0; *** p≤0.03, Teff count at Rapa doses of 26 μg versus 0 (one-way ANOVA).

FIG. 2 shows that composition 8203 preferentially attracts MMos and Treg cells to draining lymph nodes (DLN). DLN cells were counted one day after a single injection into a footpad of PBS, dexamethasone (“Dex;” 8 μg), or 8203. Data are mean±SD of four experiments. * p≤0.001 and ** p≤0.02, for absolute and relative MM counts, respectively, of 8203 injected versus any other; *** p≤0.01, relative Treg count of 8203 injected versus PBS injected (one-way ANOVA).

FIG. 3 shows the role of each component of 8203. DLN cells were counted one day after a single injection into a footpad of 8203 or a composition missing one of the three components. Data are mean±SD of four experiments. * p≤0.02 and *** p≤0.03, for absolute and relative MM$ counts, respectively, in group 2 versus any other group; ** p<0.0002 and **** p≤0.0009, absolute Teff count and relative Treg count, respectively, in group 3 versus any other group (one-way ANOVA).

FIG. 4 shows that composition 8203 allows normal uptake and processing of whole protein antigen. Mice were injected at a footpad with 5 μg of proteolytically processed DQ ovalbumin (“DQ OVA”) in either PBS or 8203; after 2 days, MMos and DCs in the DLN were analyzed for mean fluorescence intensity (MFI) resulted from intracellular cleavage of DQ OVA. AMFI=MFI from DQ OVA injected—MFI from PBS (control) injected. Data are mean±SD of four experiments. * p≤0.004 (two-sided t test).

FIG. 5 shows that composition 8203 is a complete tolerogenic adjuvant for Treg cells. Composition 8203 is shown to augments absolute expansion of KJ1-26⁺ Treg cells in DLN. The model was injected on day 5 at the primed footpad with OVA-in-PBS, OVA-in-Dex, or OVA-in-8203. DLN T cells were analyzed on day 8. The baseline of the model (on day 5) is shown as “Start.” Data are mean±SD of six (for cell count analysis) or four (for Ki67 analysis) experiments. * p≤0.001 and ** p≤0.001, for absolute and relative Treg counts, respectively, in the OVA-in-8203 injected versus any other group (one-way ANOVA). *** p≤0.04, for % Ki67⁺ Treg cells in the OVA-in-8203 injected versus any other group (one-way ANOVA).

FIG. 6 shows that all the OVA-in-8203 components are needed for absolute KJ1-26⁺ Treg cell expansion. The model was injected with indicated compositions and analyzed as in FIG. 5. Data are mean±SD of four experiments. * p<0.01 and ** p<0.001, for absolute and relative counts, respectively, of KJ1-26⁺ Treg cells in the OVA-in-8203 treated (group 2) versus any other group (one-way ANOVA).

FIG. 7 shows the role of antigen and each component of 8203 in T cell expansion. The model was injected day 5 with OVA in the components indicated once every 3 days. Expansion of KJ1-26⁺ Treg and Teff cells in indicated organs was followed in the same interval, and cell count curves generated by nonlinear regression. Each time point is mean±SD of four experiments. The day when KJ1-26⁺ T cells in the DLN reached the maximal number is defined as the “Ending day” for cell expansion. Data are mean±SD of four experiments. * p≤0.001, for Treg cells in groups 4 and 6 versus group 2 (OVA-in-8203); ** p<0.001, for Treg cells in group 7 versus group 1 (OVA-in-PBS); *** p≤0.001, for Teff cells in groups 4 and 5 versus group 2; “ns” (not significant), for Teff cells in group 7 versus group 1 (one-way ANOVA).

FIGS. 8A-8B show composition 8203-adjuvanted immunization induces antigen-specific immune suppression. (FIG. 8A) Counts of KJ1-26⁺ Treg and Teff cells in the spleen (“SP”) of immunized (“OVA-in-8203”) or control immunized (“OVA-in-PBS”) mice immediately before the challenge. Data are mean±SD of four experiments. * p=0.009 (two-sided t test). (FIG. 8B) Counts of KJ1-26⁺ Treg and Teff cells in the SP five days after the challenge. Data are mean±SD of four experiments. * p=0.03 (two-sided t test).

FIGS. 9A-9B show the effect of R848 on draining lymph node (DLN) versus non-draining lymph node (NLN). (FIG. 9A) C56BL/6 mice (10-week-old) were injected at the right-footpad with 20 μl of PBS containing DMSO (control), 3 μg of MPLA, or an equal molar amount (0.6 μg) of R848. At 24 hours, monocyte-derived macrophages (MM) in both the DLN and the NLN were counted by flow cytometry. Data are mean±SD of four experiments (on a log scale). * p≤0.001 (one-way ANOVA). (FIG. 9B) Dose-response analysis of R848. C56BL/6 mice (10-week-old) were injected subcutaneously at the back flank with 50 μl of PBS containing 8 μg of dexamethasone, 20 μg of rapamycin, and an indicated dose of R848. At 24 h, MMφ in popliteal lymph nodes were counted. Shown is a nonlinearly fitted substrate inhibition curve for mean±SD of 4-8 LNs. * p≤0.01 (one-way ANOVA).

FIG. 10 shows the effect of composition 8206 on various NLNs and the spleen. C56BL/6 mice (10-week-old) were injected subcutaneously at the back flank with 8206 or PBS. At 24 hours, MMO in popliteal (PLN), axillary (ALN), mesenteric (MLN), and caudal mediastinal (CLN) lymph nodes and the spleen (SP) were counted. Data are mean±SD of four experiments (on a log scale). *p≤0.01 (t-test).

FIG. 11 shows the effect of composition 8206 on OVA-specific Treg cells in BALB/c mice adoptively transferred with DO11.10 TCR-transgenic T cells. The mice were injected with OVA at a footpad and composition 8203 or composition 8206 at the back flank. Expansion of OVA-reactive Treg cells in the OVA-draining PLN was determined by flow cytometry. Data are mean±SD of four experiments. * p≤0.006 (one-way ANOVA) versus all other groups in Treg count.

FIG. 12 shows that Teff cells are reactive to P210m (mouse counterpart of the human apoB-100-derived peptide 210) or MOG (myelin oligodendrocyte glycoprotein35-55, as a control for antigen specificity) in indicated lymph nodes. Data are mean±SD of four experiments. * p≤0.001 versus all others in the P210m-reactive Teff count (one-way ANOVA).

FIG. 13 shows P210m-reactive Treg and Teff cells in the CLN 3 days after a single injection of composition 8206 or PBS. Data are mean±SD of six experiments. * p≤0.003 (t-test).

FIG. 14 shows HP1 (HSP 60292-308)- and HMW4 (HMGB1 98-112)-reactive T cells in the spleen 5 days after a single injection of composition 8206 or PBS. MOG was used as a control for antigen specificity. Data are mean±SD of six experiments. * p≤0.02 and ** p≤0.02 (both by t-test).

FIG. 15 shows the composition 8206 slows the growth of lesions. Average lesion coverage at the aortic root is shown. Data are mean±SD of nine mice per group (from two experiments). * p<0.001 versus all other groups (one-way ANOVA).

FIG. 16 shows cytokine levels in blood plasma. IL-2, IL-4, IL-6, IL-10, IL-17A, TNF-α, and IFN-γ were analyzed. Only TNF-α (TNF) and IFN-γ (IFN) showed a significant difference. Data are mean±SD of four mice per group. * p≤0.001 (one-way ANOVA).

### DETAILED DESCRIPTION OF THE INVENTION

A “complete” adjuvant that prevents Teff cells from reacting to antigens and expands Treg cells in absolute numbers has now been developed. As demonstrated herein, the minimum composition required for forming a vaccine adjuvant that stimulates a Treg response has now been determined, and is referred to herein as a “complete tolerogenic adjuvant.” This new adjuvant composed of dexamethasone, rapamycin, and a Toll-like receptor agonist that increases influx of monocyte-derived macrophages into lymph nodes now allows for the use of the well-proven “antigen with adjuvant” form of immunization for inducing Treg cell-mediated antigen-specific immunosuppression or targeting pathogenic auto-antigens at the site of autoimmunity.

Accordingly, provided herein is an complete tolerogenic adjuvant composition comprising an effective amount of dexamethasone, an effective amount of rapamycin, and an effective amount of a Toll-like receptor agonist that increases influx of monocyte-derived macrophages into lymph nodes. As used herein, the term “complete tolerogenic adjuvant” or “tolerogenic adjuvant” refers to a adjuvant specifically designed to promote the development of regulatory T cells (Tregs), effectively inducing immune tolerance toward a specific antigen, meaning the immune system will not mount a harmful response against it. Alternatively stated, a complete tolerogenic adjuvant is a substance that, when combined with an antigen, encourages the body to actively suppress an immune reaction against that antigen instead of attacking it.

The term “tolerance” as used herein refers to a decreased level of an immune response, a delay in the onset or progression of an immune response and/or a reduced risk of the onset or progression of an immune response. “Antigen-specific” immunological tolerance occurs when immunological tolerance is preferentially invoked against certain antigens in comparison with others.

As used herein, “regulatory T cells” or “Tregs,” or grammatical variations thereof, refer to white blood cells that regulate the immune system by controlling how it responds to substances inside and outside the body. Tregs may be characterized as described elsewhere herein and/or by expression of CD4, CD25, and FOXP3, with CD4+FOXP3+CD25^{high} Tregs generally being referred to as “naturally occurring” Tregs or nTregs to distinguish them from “suppressor” T cell populations that are generated in vitro (CD4+CD25⁺ Tregs).

Dexamethasone or 9x-fluoro-16x-methylprednisolone (CAS No. 50-02-2) is a synthetic glucocorticoid. In the complete tolerogenic adjuvant composition herein, dexamethasone converts MMos into Dex-Mos, the tolerogenic antigen presenting cells that stimulate Treg cell proliferation. In addition, together with rapamycin, dexamethasone inhibits Teff cell proliferation.

Rapamycin, also known as sirolimus or (7E, 15E, 17E, 19E)-9, 10, 12, 13, 14, 21, 22, 23, 24, 25, 26, 27, 32, 33, 34aS-Hexadecahydro-9R, 27-dihydroxy-3S-[(1R)-2-[(1S, 3R, 4R)-4-hydroxy-3-methoxycyclohexyl]-1-methylethyl]-10R, (CAS No. 53123-88-9) is a macrocyclic lactone antibiotic produced by Streptomyces hygroscopicus. In the complete tolerogenic adjuvant composition herein, rapamycin blocks the influx of Teff cells into the draining lymph node, prevents a Teff cell-dominated response, and/or prolongs Treg cell expansion.

Toll-like receptors (TLRs) are a class of proteins that play a key role in the innate immune system. A “Toll-like receptor agonist,” “TLR agonist,” “Toll-like receptor ligand,” or “TLR ligand” is a substance that binds to and activates at least one TLR. It is noted that there are differences between humans and mice as to which TLR may be activated by any given agonist, and this must be taken into account depending upon the species used. As used herein, a TLR agonist may include, but is not limited to a TLR2/1 agonist, TLR2/6 agonist, TLR3 agonist, TLR4 agonist, TLR5 agonist, TLR7 agonist, TLR8 agonist, TLR7/8 agonist, TLR9 agonist, TLR10 agonist, and/or TLR11 agonist.

Bacterial lipopeptides are the main agonists for TLR2-containing receptors. These agonists include, e.g., mycoplasmal macrophage-activating lipoprotein-2; tripalmitoyl-cysteinyl-seryl-(lysyl) 3-lysine (P3-CSK4; CAS No. 112208 Jan. 2); dipalmitoyl-CSK4 (P2-CSK4; CAS No. 574741-81-4); monopalmitoyl-CSK4 (PCSK4); tripalmitoyl-S-glyceryl-cysteine (Pam (3) Cys)-modified lipoproteins, including OspA from the Lyme disease spirochete Borrelia burgdorferi; mycobacterial cell wall fractions enriched for lipoarabinomannan, mycolylarabinogalactan-peptidoglycan complex, or M. tuberculosis total lipids.

TLR3 agonists may be activated by viral double stranded RNAs (dsRNAs), endogenous messenger RNA (tnRNA), bacterial RNA, and/or polyriboinosinic-polyribocytidylic acid or Poly (I: C) which is a synthetic form of dsRNA.

The classic agonist for TLR4 is bacterial lipopolysaccharide (LPS), which refers to a family of substances containing lipid A and its congeners. An exemplary form of LPS is E. coli B: 0111 (Sigma Chemicals). However, in an effort to make a less toxic form of TLR4 agonist, monophosphoryl lipid A (MPLA; CAS No. 1246298-63-4) compounds have been produced.

The principal agonist for TLR5 is bacterial flagellin.

TLR7 agonists include, but are not limited to, single-stranded RNA; imidazoquinoline compounds such as resiquimod (R848; CAS No. 144875-48-9) and imiquimod (CAS No. 99011 Feb. 6); Loxoribine (7-allyl-7,8-dihydro-8-oxo-guanosine; CAS No. 121288-39-9) and related compounds; 7-Thia-8-oxoguanosine (CAS No. 122970-43-8), 7-deazaguanosine (CAS No. 62160-23-0), and related guanosine analogs; ANA975 (CAS No. 847453-47-8) and related compounds; SM-360320 (CAS No. 226907-52-4); Telratolimod (3M-052, CAS No. 1359993-59-1) or 3M-011 (CAS No. 642473-62-9); or Guretolimod (DSP-0509; CAS No. 1488364-57-3). Many of the compounds that activate TLR7 also activate TLR8. Poly-G containing 10 guanosine nucleosides connected by phosphorothioate linkages (Poly-G10) is also a TLR8 agonist.

Oligonucleotides or polynucleotides such as CpG-containing oligodeoxynucleotides (CpG ODN) are the prototype agonists for TLR9. Typically, the ODN is a synthetic thiophosphorylate-linked compound. However, many types of DNA and RNA may activate TLR9 including bacterial DNA, liposomal vertebrate DNA, insect DNA, chlamydia polynucleotides and others. Another class of TLR9 agonists are nucleotide sequences containing a synthetic cytosine-phosphate-2′-deoxy-7-deazaguanosine dinucleotide (CpR). In addition, a dumbbell-like covalently-closed known as lefitolimod is an agonist for TLR9.

An agonist for TLR11 may be a profilin-like molecule from the protozoan parasite Toxoplasma gondii (PFTG).

In some aspects, a TLR agonist of use in the complete tolerogenic adjuvant composition herein is one that increases influx of monocyte-derived macrophages into lymph nodes as compared to a tolerogenic adjuvant composition lacking the TLR agonist. In some aspects, the TLR agonist is a TLR4, TLR7, and/or TLR8 agonist. In some aspects, the TLR agonist that increases influx of monocyte-derived macrophages into lymph nodes is MPLA or resiquimod (R848).

In one aspect, the dexamethasone, rapamycin, and Toll-like receptor agonist are included in the complete tolerogenic adjuvant composition in a mass ratio in the range of about 6 to about 10 dexamethasone (e.g., about 6 to about 10, about 7 to about 9 dexamethasone): about 15 to about 25 rapamycin (e.g., about 15 to about 25, about 17 to about 22 rapamycin): about 1 to about 8 Toll-like receptor agonist (e.g., about 1 to about 8, about 2 to about 7 rapamycin). In some aspects, the dexamethasone, rapamycin, and Toll-like receptor agonist of the complete tolerogenic adjuvant composition are in a mass ratio of about 8:20:3 or about 8:20:6.

In one aspect, the complete tolerogenic adjuvant composition comprises, consists of, or consists essentially of dexamethasone, rapamycin, and monophosphoryl lipid A in a mass ratio of about 8:20:3. In another aspect, the complete tolerogenic adjuvant composition comprises, consists of, or consists essentially of dexamethasone, rapamycin, and resiquimod in a mass ratio of about 8:20:6.

In some aspects, the complete tolerogenic adjuvant is in the form of a vaccine. As used herein, a “vaccine” is defined as an immunostimulatory composition designed to elicit an immune response against an antigen, whether administered prophylactically or for the treatment of an already existing condition. In some aspects herein, vaccination or immunization may decrease a recipient's immune response against self-antigens, allergens, and/or allo-antigens thereby respectively decreasing the likelihood of an autoimmune response, allergic response, and/or graft rejection. “Immune response” generally refers to innate and acquired immune responses including, but not limited to, both humoral immune responses (mediated by B lymphocytes) and cellular immune responses (mediated by T lymphocytes). An immune response may be beneficial and lead to immunity against infectious pathogens, or an immune response may be pathogenic and lead to autoimmune or hypersensitivity disease. Immune responses against foreign viruses, bacteria, fungi, and parasites typically represent beneficial adaptive immune responses. Immune responses against self-tissues, innocuous foreign objects (e.g., dust mite or pollen allergens, etc.), or tissue transplants represent examples of adverse maladaptive immune responses.

In some aspects, a vaccine includes a complete tolerogenic adjuvant as described here and a pharmaceutically acceptable carrier, diluent, or excipient. A “pharmaceutically acceptable carrier, excipient, or diluent” refers to any substance suitable for delivering a vaccine described herein that is compatible with the other ingredients of the formulation, and not deleterious to the recipient thereof. Carriers that may be used with a vaccine herein include, but are not limited to, e.g., water, phosphate-buffered saline, Ringer's solution, dextrose solution, serum-containing solutions, Hank's solution, and other aqueous physiologically balanced solutions. See, for example, Remington: The Science and Practice of Pharmacy, most recent edition.

In some aspects, a vaccine comprises, consists of, or consists essentially of a complete tolerogenic adjuvant as described here in combination with a pharmaceutically acceptable carrier, and is devoid of any antigen. In some aspects, a vaccine comprises, consists of, or consists essentially of a complete tolerogenic adjuvant as described here, a pharmaceutically acceptable carrier, and further includes an antigen. The term “antigen” as used herein means a substance or compound that stimulates an immune response. Although usually a protein or polysaccharide, antigens may be any type of molecule, which may include small molecules (haptens) that are coupled to a carrier-protein. In some aspects, the antigen is one or more of an autoimmune antigen, allergen, or alloantigen.

As used herein, term the “autoimmune antigen,” “autoantigen,” or grammatical variations thereof, refers to any self-protein or self-component that serves either as a target or cause of an autoimmune disease. Examples of autoimmune antigens include, but are not limited to, myelin basic protein, proteolipid protein, or myelin oligodendrocyte protein (multiple sclerosis); peripheral myelin proteins P0 and P2 (Guillain-Barre syndrome); acetylcholine receptor (myasthenia gravis); cardiac myosin (rheumatic fever/myocarditis); proteins of the beta cells in the Isles of Langerhans-GAD (glutamic acid decarboxylase), insulin (Type I autoimmune diabetes mellitus), the thyroid-stimulating hormone receptor (Grave's disease), platelets (thrombocytopenia purpura), neuromuscular junction (myasthenia gravis), red blood cells (autoimmune hemolytic anemia) intracellular antigens (spliceosomes, ribosomes, nucleic acid, etc. in systemic lupus erythematosus), tyrosinase-related protein-1 (vitiligo), and apolipoprotein B-100 (atherosclerosis). In some aspects, the autoimmune antigen is a neuroantigen, i.e., a type of autoimmune antigen that is a nervous system protein (central or peripheral) including an auto-reactive epitope. The neuroantigen may be a myelin basic protein (MBP), a proteolipid protein (PLP), myelin oligodendrocyte glycoprotein (MOG), myelin-associated oligodendrocytic basic protein (MOG), or other nervous system-derived proteins or a portion thereof and further including those derived from any species, and in particular, human, rat, mouse, goat and sheep.

An “allergen” refers to any substances that can cause an undesired (e.g., a Type 1 hypersensitive) immune response (i.e., an allergic response or reaction) in a subject. Allergens include, but are not limited to, plant allergens (e.g., pollen, ragweed allergen), insect allergens, insect sting allergens (e.g., bee sting allergens), animal allergens (e.g., pet allergens, such as animal dander or cat Feld1 antigen), latex allergens, mold allergens, fungal allergens, cosmetic allergens, drug allergens, food allergens, dust, insect venom, viruses, bacteria, etc. Food allergens include, but are not limited to milk allergens, egg allergens, nut allergens (e.g., peanut or tree nut allergens such as walnuts, cashews, etc.), fish allergens, shellfish allergens, soy allergens, legume allergens, seed allergens and wheat allergens. Insect sting allergens include allergens that are or are associated with bee stings, wasp stings, hornet stings, yellow jacket stings, etc. Insect allergens also include house dust mite allergens (e.g., Der P1 antigen) and cockroach allergens. Drug allergens include allergens that are associated with antibiotics, NSAIDS, anesthetics, etc. Pollen allergens include grass allergens, tree allergens, weed allergens, flower allergens, etc.

“Alloantigen” refers to an antigen present only in some individuals of a species and capable of inducing the production of an alloantibody by individuals which lack it. Examples thereof are MHC class I and/or class II molecules, minor histocompatibility antigens, and tissue-specific alloantigens.

Having demonstrated that a complete tolerogenic adjuvant as described herein can induce a Treg cell-mediated antigen-specific immunosuppressive response, whether administered in combination with an antigen or as an antigen-less composition, some aspects provide for a method of inducing regulatory T cell-mediated antigen-specific immunosuppression in a subject in need thereof by administering to the subject an effective amount of a complete tolerogenic adjuvant composition comprising dexamethasone, rapamycin, and a Toll-like receptor agonist that increases influx of monocyte-derived macrophages into lymph nodes (e.g., a TLR7/8 agonist or a TLR4 agonist).

In some aspects, the complete tolerogenic adjuvant composition is devoid of any antigen and/or administered in the complete absence of any therapeutically administered antigen (e.g., a therapeutically administered autoantigen). In some aspects, the complete tolerogenic adjuvant composition is administered in combination with an antigen, either in the same composition and/or by co-administration, e.g., either simultaneously or sequentially. In some aspects, the antigen may be associated with a disease or condition described herein. An “antigen associated” with a disease or condition provided herein are antigens that may generate an undesired immune response against, as a result of, or in conjunction with, the disease or condition; the cause of the disease or condition (or a symptom or effect thereof); and/or may generate an undesired immune response that is a symptom, result or effect of the disease or condition.

In some aspects, the subject is administered a complete tolerogenic adjuvant composition comprising dexamethasone, rapamycin, and resiquimod in a mass ratio of 8:20:6, respectively. In some aspects, the subject is administered a complete tolerogenic adjuvant composition comprising dexamethasone, rapamycin, and MPLA in a mass ratio of 8:20:3, respectively, optionally in combination with an antigen associated with a disease or condition.

In some aspects, the immune response being suppressed by Tregs is an antigen-specific Teff response. As used herein, “effector T cell” or “Teff” refers to T cells which are not regulatory and which have encountered antigen and costimulatory molecules. Effector T cells may be characterized as described elsewhere herein and/or by certain markers of activation, e.g., cytokine production, and/or biomarker expression, e.g., CD4⁺.

In some aspects, the administration of an effective amount of a complete tolerogenic adjuvant as described herein may result in at least about a 2-fold (e.g., about a 2-, 3-, 4-, 5-, 6-, 7-, 8-, 9-, 10-fold or more) decrease in the number of antigen-specific Teff in the subject (e.g., in one or more lymph nodes of the subject) as compared a control subject, e.g., the subject prior to treatment with the complete tolerogenic adjuvant or a subject with a similar immune response that has not received treatment with the complete tolerogenic adjuvant. In some aspects, the administration of a complete tolerogenic adjuvant as described herein may result in at least about a 2-fold to about a 10-fold, or at least about a 5-fold to about a 10-fold decrease in the number of antigen-specific Teff in the subject as compared to a control subject.

In some aspects, the administration of an effective amount of a complete tolerogenic adjuvant as described herein may result in at least about a 2-fold (e.g., about a 2-, 3-, 4-, 5-, 6-, 7-, 8-, 9-, 10-fold or more) increase in the number of Tregs in the subject (e.g., in one or more lymph nodes of the subject) as compared a control subject, e.g., the subject prior to treatment with the complete tolerogenic adjuvant or a subject with a similar immune response that has not received treatment with the complete tolerogenic adjuvant. In some aspects, the administration of a complete tolerogenic adjuvant as described herein may result in at least about a 2-fold to about a 10-fold, or at least about a 5-fold to about a 10-fold increase in the number of Tregs in the subject as compared to a control subject.

Teff and/or Tregs may be monitored using, for example, methods of assessing immune cell number and/or function, tetramer analysis, ELISPOT, flow cytometry-based analysis of cytokine expression, cytokine secretion, cytokine expression profiling, gene expression profiling, protein expression profiling, analysis of cell surface markers, PCR-based detection of immune cell receptor gene usage (see, e.g., Clay et al. (2001) Clin. Cancer Res. 7:1127-1135). In addition, specific methods are described in more detail in the Examples.

Also provided herein are methods of treating or reducing the risk of developing an autoimmune disease or condition, allergy, an inflammatory disease, transplant rejection or graft versus host disease by administering to a subject in need thereof an effective amount of a complete tolerogenic adjuvant composition comprising dexamethasone, rapamycin, and a Toll-like receptor agonist that increases influx of monocyte-derived macrophages into lymph nodes (e.g., a TLR7/8 agonist or a TLR4 agonist). In some aspects, the complete tolerogenic adjuvant composition is devoid of an antigen and/or administered in the complete absence of an exogenously administered antigen, e.g., an autoimmune antigen associated with the autoimmune disease or condition. In some aspects, the subject is administered a complete tolerogenic adjuvant composition comprising dexamethasone, rapamycin, and resiquimod in a mass ratio of 8:20:6, respectively. In some aspects, the complete tolerogenic adjuvant composition is administered in combination with antigen as described herein, either in the same composition and/or by co-administration, either simultaneously or e.g., sequentially. In some aspects, the subject is administered a complete tolerogenic adjuvant composition comprising dexamethasone, rapamycin, and MPLA in a mass ratio of 8:20:3, respectively, optionally in combination with an antigen.

As used herein, the administration of a complete tolerogenic adjuvant “in conjunction with” or “in combination with” an antigen means that the substances are administered closely enough in time that the presence of one alters the biological effects of the other. The two substances may be administered in conjunction simultaneously (i.e., concurrently) or sequentially. Simultaneous administration may be carried out by mixing the substances prior to administration and providing the same as a mixture, by administering the substances at the same point in time but at different anatomic sites or using different routes of administration. Sequential administration may be carried out by administering one of the substances prior to or before the other, and consequently, administering one of the substances after the other.

Subjects that may be treated with any of the complete tolerogenic adjuvant compositions and methods provided herein include those who have or are at risk of having an autoimmune disease or condition, those who have or are at risk of having an allergy to any of the allergens provided herein, and/or those who have undergone or will undergo transplantation. The methods herein are also useful for promoting tolerogenic immune responses in subjects that have received, are receiving or will receive a therapeutic protein against which undesired immune responses are generated or are expected to be generated. The compositions and methods herein may be used to prevent or suppress undesired immune responses that may neutralize the beneficial effect of certain therapeutic treatments. The compositions and methods herein may be used to inhibit, suppress or delay the development, progression or pathology of the diseases, disorders or conditions described herein. Whether or not a complete tolerogenic adjuvant composition described herein can lead to the inhibition of the development, progression or pathology of the diseases, disorders or conditions described herein may be measured with animal models of such diseases, disorders or conditions. In some aspects, the reduction of an undesired immune response or generation of a tolerogenic immune response may be assessed by determining clinical endpoints, clinical efficacy, clinical symptoms, disease biomarkers and/or clinical scores. Undesired immune responses or tolerogenic immune responses may also be assessed with diagnostic tests to assess the presence or absence of a disease, disorder or condition as provided herein. For example, methods for monitoring or assessing undesired allergic responses include assessing an allergic response in a subject by skin reactivity and/or allergen-specific antibody production.

A subject in need of treatment may be an animal, including warm blooded mammals such as humans and primates; avian; domestic household or farm animal such as cats, dogs, sheep, goats, cattle, horses and pigs; laboratory animal such as mice, rats and guinea pigs; fish; reptile; zoo and wild animal; and the like.

In the methods herein, a pharmaceutical composition or vaccine comprising a complete tolerogenic adjuvant may be administered via any suitable route including, but not limited to, parenteral, intraarterial, intranasal or intravenous administration or by injection to lymph nodes or anterior chamber of the eye or by local administration to an organ or tissue of interest. In some aspects, the administering is by subcutaneous, intrathecal, intraventricular, intramuscular, intraperitoneal, intracoronary, intrapancreatic, intrahepatic or bronchial injection.

As used herein, the term “treating,” “treat,” or “treatment” refers to reducing, ameliorating, or eliminating in a subject having a disease or condition a clinical symptom of the disease or condition. The term “reducing the risk of developing” refers to a delay or suppression of the onset of a clinical symptom of a disease or condition. For example, the term “treating” may mean reducing a symptom of a disease or condition by, e.g., at least 20%, at least 30%, at least 40%, at least 50%, at least 60%, at least 70%, at least 80%, at least 90% or at least 100%.

aspects, an effective amount of a complete tolerogenic adjuvant as described herein refers to the amount of the complete tolerogenic adjuvant that will elicit the biological or clinical response being sought by the practitioner in an individual in need thereof. As a non-limiting example, an effective amount is an amount sufficient to reduce a symptom of an autoimmune disease or condition, allergy, or graft rejection including, e.g., edema, hyperemia, erythema, bruising, tenderness, stiffness, swollenness, fever, chills, stuffy nose, stuffy head, breathing difficulties, fluid retention, blood clots, loss of appetite, increased heart rate, formation of granulomas, fibrinous, pus, non-viscous serous fluid, ulcer, increased production of self-reactive effector immune cells, inflammation, or pain.

The appropriate effective amount to be administered for a particular application of the disclosed methods may be determined by those skilled in the art, using the guidance provided herein. For example, the effectiveness of a complete tolerogenic adjuvant disclosed herein in treating a symptom of a disease or condition described herein may be determined by observing one or more clinical symptoms, and/or physiological indicators associated with the condition. The response of an individual with a disease or condition to treatment may be monitored by determining the severity of their symptoms or by determining the frequency of Teff and/or Treg cells in a sample from an individual with an disease or condition.

A complete tolerogenic adjuvant composition herein may be administered to a subject in need thereof in the form of a pharmaceutical composition or vaccine in one or more doses, e.g., 1, 2, 3, 4, 5, 6, 7, 8, 9, 10, 11, 12, 13 or 14 doses. In some aspects, a subject may receive at least 2 doses including, e.g., between 2-50 doses, more particularly between 2-28 doses, and more particularly between 2-14 doses. In some aspects, the at least two may be separated by any suitable amount of time, e.g., hours, days or weeks.

An complete tolerogenic adjuvant composition herein may be administered to a subject in need thereof in the form of a pharmaceutical composition or vaccine at a dose of 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg per kg of body weight.

In some aspects, compositions and methods herein may be used to treat or reduce the risk of developing an autoimmune disease or condition. The terms “autoimmune disease” or “autoimmune condition” refer to diseases that result from an aberrant immune response of a subject against its own cells and tissues due to a failure of the subject to recognize its own constituent parts (down to the sub-molecular level) as “self.” Examples of antigens involved in autoimmune diseases include, e.g., thyroglobulin, thyroid peroxidase, TSH receptor, insulin (proinsulin), glutamic acid decarboxylase (GAD), tyrosine phosphatase IA-2, myelin oligodendrocyte protein, and heat-shock protein HSP60. Autoimmune diseases include, but are not limited to, those affecting biological systems such as the circulatory system, digestive system, endocrine system, integumentary system, lymphatic system, muscular system, nervous system, reproductive system, respiratory system, skeletal system or urinary system. In particular, the biological systems may include the nervous system: Acute disseminated encephalomyelitis (demyelinating inflammation following vaccination or infection); Myasthenia Gravis (anti-AchR antibodies, blockade of neuromuscular junction); Multiple sclerosis (inflammation of CNS myelin); Acute inflammatory demyelinating polyneuropathy/Guillain-Barre syndrome (inflammation of peripheral myelin); Endocrine system: Hashimoto's Thyroiditis (anti-thyroid antibodies, hypothyroidism); Grave's Disease (auto-antibodies stimulate TSH receptors on thyroid follicular cells, hyperthyroidism); Insulin-Dependent Diabetes Mellitus (i.e., juvenile diabetes, inflammation and deletion of p islet cells); autoimmune adrenal insufficiency (e.g., Addison's disease, inflammation coupled with progressive scarring and atrophy of adrenal glands); autoimmune oophoritis (inflammation of ovaries, infertility); autoimmune orchitis (inflammation of testis); hematopoietic system: autoimmune hemolytic anemia (anti-erythrocyte antibodies); paroxysmal cold hemoglobinuria (mediated by IgM cold agglutinins against erythrocytes); idiopathic thrombocytopenia purpura (anti-platelet antibodies, bleeding); autoimmune neutropenia (antibodies against neutrophils cause degranulation, neutrophil depletion, and vasculitis); pernicious anemia (progressive destruction of gastric fundic gland, loss of intrinsic factor, and malabsorption of vitamin B12); autoimmune coagulopathy (circulating anti-coagulants, anti-phospholipid antibody syndrome, neutralizes phospholipids necessary for clotting activity); gastrointestinal tract: primary biliary cirrhosis (intrahepatic bile duct and portal inflammation leading to fibrosis and cirrhosis); inflammatory bowel disease (Crohn's disease, ulcerative colitis); kidney: glomerulonephritis (antibody against glomerular basement membrane); immune complex glomerular nephritis (accumulation of deposited immune complexes in basement membrane); skin: Pemphigus vulgaris (loss of adhesion between epidermal cells, blistering, antibody against stratified squamous epithelium); systemic autoimmune disease: Systemic Lupus Erythematosus (arthralgias, rash, nephritis, anti-nuclear antibodies); rheumatoid arthritis (inflammatory polyarticular arthritis, rheumatoid factor); Sjogren's syndrome (inflammation of lacrymal and parotid glands with arthritis); polymyositis (inflammation of skeletal muscle); dermatomyositis (inflammation of skin and skeletal muscle); scleroderma (progressive systemic sclerosis, sclerosis of skin and internal organs); and cardiac and vascular diseases: autoimmune myocarditis (inflammation of cardiac muscle); immune complex-mediated vasculitis (passive deposition of immune complexes in vessel walls followed by C-mediated lysis and inflammation); polyarteritis nodosa (type of necrotizing vasculitis that follows certain types of infections). In some aspects, the autoimmune disease is an autoimmune disease affecting the nervous system, endocrine system, hematopoietic system, gastrointestinal tract, renal system, cardiac system, vascular system, musculoskeletal system, or a combination thereof. In some aspects, the autoimmune disease is a systemic autoimmune disease.

In some aspects, compositions and methods herein may be used to treat or reduce the risk of developing an allergy. As used herein, an “allergy” also referred to herein as an “allergic condition” or “hypersensitivity disease” is any condition where there is an undesired immune response to an allergen. Allergies or allergic conditions include, but are not limited to, allergic asthma, hay fever, hives, eczema, plant allergies, bee sting allergies, pet allergies, latex allergies, mold allergies, cosmetic allergies, food allergies, allergic rhinitis or coryza, topic allergic reactions, anaphylaxis, atopic dermatitis, hypersensitivity reactions and other allergic conditions. The allergic reaction may be the result of an immune reaction to any allergen. In some aspects, the allergy is a food allergy. Food allergies include, but are not limited to, milk allergies, egg allergies, nut allergies, fish allergies, shellfish allergies, soy allergies or wheat allergies.

In some aspects, compositions and methods herein may be used to treat or reduce the risk of developing transplant rejection (e.g., organ or tissue rejection) or graft versus host disease. Transplant rejection and tissue disorders include, but are not limited to, those affecting the appendix, bladder, brain, ear, esophagus, eye, gall bladder, heart, kidney, large intestine, liver, lung, mouth, muscle, nose, ovary, pancreas, parathyroid gland, pineal gland, pituitary gland, skin, small intestine, spleen, stomach, testes, thymus, thyroid gland, trachea, uterus, vermiform appendix or combinations thereof. In some aspects, a complete tolerogenic adjuvant composition and method herein may be used to induce antigen-specific immunological tolerance to allogeneic and xenogeneic transplantation antigens that may contribute to the rejection of tissue transplants, and thus, facilitate acceptance of kidney transplants, liver transplants, pancreas transplants, skin grafts, heart transplants, and heart-lung transplant or other organs listed above. The complete tolerogenic adjuvant and methods may also alleviate complications of bone marrow transplantation (i.e., graft versus host disease).

The following non-limiting examples are provided to further illustrate the present invention.

### EXAMPLE 1: Complete Tolerogenic Adjuvant Stimulates Treg Response to Immunization

Mice. Use of mice was approved by the Biologic Resource Committee of University of Illinois College of Medicine at Rockford. BALB/c Foxp3-eGFP and C57BL/6 Foxp3-eGFP mice were originally purchased from the Jackson Laboratory. BALB/c DO11. 10 TCR-transgenic Foxp3-eGFP (“DO11.10 Foxp3-eGFP”) mice were generated by crossing BALB/c Foxp3-eGFP mice with BALB/c DO11.10 TCR-transgenic mice (from the Jackson Laboratory). All mice were used at age of 8 weeks unless indicated otherwise. Female and male mice were used in equal numbers in separate groups. Although sex difference was noted in the baseline counts of CD4⁺ T cells and CD11cleCD40l monocyte-derived macrophages (“MMos”) in lymph nodes and the spleen, the difference was modest and did not change the responses of the mice to the adjuvants tested. Consequently, all the data reported were pooled from both female and male groups; and the word “mice” is used to indicate the inclusion of both sexes.

Reagents. Dexamethasone sodium phosphate was from ValleyVet. Rapamycin (dissolved in DMSO first) and FTY720 (fingolimod HCl; dissolved in DMSO first) were from Selleckchem. DQ ovalbumin, sodium heparin, ACK lysing buffer, FICOLL-Paque Plus, Freund's complete adjuvant (“FCA”), and Freund's incomplete adjuvant (“FIA”) were from ThermoFisher. Hen ovalbumin (“OVA”), hen egg lysozyme (“HEL”), and monophosphoryl lipid A (“MPLA;” dissolved in DMSO first) were from Sigma-Aldrich. Protease- and Ig-free bovine serum albumin (BSA) was: from SouthernBiotech. Antibodies for flow cytometry were from Biolegend, eBioscience, and Jackson ImmunoResearch Laboratories. Cell counting beads for flow cytometry were from Bangs Laboratories. Mouse Th1/Th2/Th17 CBA kit was from BD Biosciences. Clodronate liposomes and control liposomes were a gift from Roche Diagnostics GmbH. Mouse CD4⁺ T cell isolation kit (negative selection), mouse CD4+CD25⁺ regulatory T cell isolation kit, and mouse CD3& MicroBead kit were from Miltenyi Biotec. eFluor® 670 fluorescent dye was from eBioscience (dissolved in DMSO first). The peptide OVA323-339 was from AnaSpec.

Footpad Injection. “A footpad” means the footpad of the right hind leg. Adjuvant components, antigens, or their combinations were injected subcutaneously (s.c.) between the 3rd and 4th toes in a volume of ˜20 μl in phosphate-buffered saline (PBS) with a BD insulin syringe.

Dose-Response Analysis. For dose-response analysis of MPLA, the reagent was serially diluted in DMSO; 5 μl of the diluted reagent were mixed with 100 μl of PBS and the resulting mixture was injected at 20 μl/footpad. For dose 0, 5 μl of DMSO were mixed with 100 μl of PBS and the resulting mixture was injected. Dose-response analysis of rapamycin was performed similarly, except that PBS containing 3 μg of MPLA was used in place of PBS.

Preparation of Single Cell Suspension from Lymphoid Organs. Lymph nodes or the spleen from studied mice were placed in a 1.5 ml microtube containing blood buffer (PBS/0.5% protease- and Ig-free BSA/2 mM EDTA); in the tube, the organs were cut with 4-inch fine scissors for 200 times and then homogenized gently with a microtube sample pestle. Released cells were filtered through an SP Bel-Art Flowmi 70 μM cell strainer.

Analysis of Adjuvant-Induced Potentiation in Lymphoid Organs. A cell suspension was prepared from the draining lymph node (“DLN,” popliteal), non-draining lymph nodes (“NLNs,” contralateral brachial and axillary nodes), or the spleen, as described above. For counting MMos and dendritic cells (“DCs”), a portion of the cell suspension was stained with biotin anti-CD3, biotin anti-CD19, PE/Cy7 APC anti-CD40, and PE streptavidin in the presence of mIgG as a blocker. Cells were analyzed by flow cytometry. PE+ cells were gated out. The total counts of MMos (CD11^{lo}CD40^{lo}) (Zheng et al. (2013) Eur. J. Immunol. 43:219-227) and DCs (all the CD11+CD40⁺ cells after excluding the CD11^{lo}CD40^{lo} cells) (Zheng et al. (2013) Eur. J. Immunol. 43:219-227) in the cell suspension were determined by the method of Schlenke et al. ((1998) Cytometry 33:310-317). The MMφ count in percent was calculated as % of the total MMO and DC counts combined.

For counting Treg and Teff cells, a portion of the cell suspension was stained with PE/Cy7 anti-CD3 and APC anti-CD4 and analyzed by flow cytometry. total The counts of Treg (CD3+CD4+Foxp3-eGFP+) and Teff (CD3+CD4+Foxp3-eGFP-) cells in the suspension were determined by the method of Schlenke et al. ((1998) Cytometry 33:310-317). The Treg count in percent was calculated as % of the total Treg and Teff counts combined.

Preparation of 8203. Composition 8203 was prepared in the amount of ten doses as follows: 27 μl of dexamethasone sodium phosphate (3 μg dexamethasone/μl) were added to 165 μl of PBS in a microtube. Next, 3 μl of rapamycin (60 μg/μl in DMSO) were added, and the tube was vortexed at the top speed for 13 seconds, which produced a cloudy solution. Lastly, 5 μl of MPLA (6 μg/μl in DMSO) were added and mixed. The final solution remained cloudy and was used at 20 μl/dose.

Mouse Model Testing Tolerogenic Adjuvants (“the Model”). The model was generated as follows: On day 0, whole blood was collected with heparin as the anticoagulant from a DO11.10 Foxp3-eGFP mouse. The blood was diluted 1:50 in PBS and transfused intravenously (i.v.) into sex-matched syngeneic BALB/c Foxp3-eGFP mice at ˜100 μl (containing ˜4,000 CD4+KJ1-26⁺ cells) per mouse. On day 1, the recipients were injected at a footpad with 100 μg of OVA in 20 μl of PBS (see “Footpad Injection”) and let rest. On day 5, they were used as the model.

To determine the origin of OVA-in-8203-expanded Treg cells, an alternative model was generated. Briefly, blood was collected by cardiac puncture from DO11.10 Foxp3-eGFP mice. Leukocytes were isolated via FICOLL-Paque density gradient centrifugation, from which CD4⁺ Teff cells were enriched untouched using the Miltenyi mouse CD4+CD25⁺ regulatory T cell isolation kit. The enriched cells were flow sorted twice with a FACS Melody sorter, excluding any Treg (Foxp3-eGFP+) cells. The purified Teff cells were transfused (i.v.) into BALB/c Foxp3-eGFP mice at 4,000 cells/mouse (day 0). The recipient mice were primed on day 1 and used on day 5, as described above.

Determination of CD4⁺ T cell Counts in the Model. A cell suspension was prepared from the DLN, NLNs, or the spleen as described herein. For counting CD4⁺ T cells in the DLN, a portion of the DLN cell suspension was stained with PE/Cy7 anti-CD3 and APC anti-CD4 and analyzed by flow cytometry. The total CD4⁺ T cell count (CD3+CD4+) in the suspension was determined by the method of Schlenke et al. ((1998) Cytometry 33:310-317). Another portion was stained with biotin anti-F4/80, biotin anti-CD8, biotin anti-CD19, PE KJ1-26 (anti-DO11.10 TCR), APC anti-CD3, and PE/Cy7 streptavidin in the presence of mIgG as a blocker. Cells were analyzed by flow cytometry. The total counts in the suspension for KJ1-26⁺ Treg, KJ1-26⁺ Teff, host Treg, and host Teff cells were calculated as portions of the total CD4⁺ T cell count. The count in percent for Treg cells (“% Treg”) was calculated as % of the total Treg and Teff counts combined.

Cell suspensions prepared from NLNs and the spleen contained far more cells. To shorten the time needed for flow cytometry, irrelevant cells were removed from the suspension with the Miltenyi CD4⁺ T cell isolation kit after the total CD3+CD4⁺ cell count in the suspension was determined. The rest of the steps were the same as described before.

Analysis of CD4⁺ T Cell Proliferation in the Model. A cell suspension was prepared from the DLN, NLNs, or the spleen as described herein and stained with biotin anti-F4/80, biotin anti-CD8, biotin anti-CD19, PE/Cy7 anti-CD3, and APC KJ1-26 in the presence of mIgG as a blocker. Biotint cells were depleted with anti-biotin MACS beads (from the mouse CD4⁺ T cells isolation kit). The remaining cells were fixed in 4% paraformaldehyde, permeabilized in 0.5% TWEEN-20, stained with PE anti-Ki67 (clone SolA15 from eBioscience) in the presence of mIgG, and analyzed by flow cytometry. The count in percent for Ki67⁺ cells (“% Ki67+”) was calculated as % of Ki67⁺ and Ki67-cells combined.

Preparation of OVA-in-8203. OVA-in-8203 was prepared in the amount of ten doses by mixing 10 μl of OVA (100 μg/μl in PBS) with ten doses of 8203 (200 μl). The final solution was used at 21 μl/dose. As controls, ten doses of OVA-in-Dex were prepared by mixing 10 μl of OVA, 27 μl of dexamethasone sodium phosphate (3 μg dexamethasone/μl), and 173 μl of PBS; and ten doses of OVA-in-PBS by mixing 10 μl of OVA with 200 μl of PBS.

Clodronate Liposome Treatment. The model was injected (i.v.) with clodronate liposomes or plain liposomes (control) on day 4 (150 μl/mouse), day 5 (100 μl/mouse), and day 6 (100 μl/mouse) (defined in “Mouse Model for Testing Tolerogenic Adjuvants”). The mice were also injected at the primed footpad with OVA-in-8203 on day 5. MMos in the DLN were counted (see “Analysis of Adjuvant-Induced Potentiation in Lymphoid Organs”) on days 6-8. KJ1-26⁺ Treg cells in the same DLN were counted (see “Analysis of CD4⁺ T Cell Counts in the Model”) on day 8.

Inhibition of T Cell Egress with FTY720. FTY720 was dissolved in DMSO at 20 μg/μl. On day 5 in the model, 1 μl of FTY720 was mixed with one dose of OVA-in-8203, and the mixture was injected into the primed footpad. As a control, 1 μl of DMSO was used in place of FTY720, and the mixture was injected into a control group. On days 6 and 7, the mice were given additional injections (i.p.) of FTY720 (or DMSO) at 1 μl (diluted in 100 μl PBS)/mouse/day. On day 8, KJ1-26⁺ Treg and Teff cells in the DLN, NLNs, and the spleen were counted, as described earlier.

Analysis of Treg Markers. A cell suspension was prepared from pooled DLNs from the model that had been given an optimal regime of OVA-in-8203. The cells were stained with biotin anti-F4/80, biotin anti-CD8, biotin anti-CD19, PE/Cy7 anti-CD3, and APC KJ1-26 in the presence of mIgG as a blocker. Biotin+ cells were depleted with anti-biotin MACS beads. The remaining cells were stained with one of the following PE-labeled mAbs: anti-CD25, anti-CTLA4, anti-PD1, anti-TNFR2, and anti-4-1BB. Some of the cells were fixed/permeabilized and intracellularly stained with PE anti-IL-10. The results were analyzed by flow cytometry. KJ1-26⁺ Treg cells (CD3+KJ1-26+Foxp3-eGFP+) were gated. The same analysis was performed on naïve KJ1-26⁺ Treg cells (from pooled popliteal lymph nodes of naïve DO11. 10 Foxp3-eGFP mice) as a reference.

Suppression Assay. Splenocytes were isolated from BALB/c mice, depleted of T cells with the anti-CD3& MicroBead kit, and used as stimulators. Untouched Teff cells were isolated from pooled lymph nodes of naive DO11.10 Foxp3-eGFP mice with the mouse CD4+CD25⁺ regulatory T cells isolation kit, stained with eFluor® 670 (Zheng et al. (2013) Eur. J. Immunol. 43:219-227), and used as responders. Naïve “source Treg cells” (“Tregl”) were isolated along with the Teff cells, from which the Foxp3-eGFP+ fraction was purified using a FACS Melody sorter. Expanded source Treg cells (“Treg²”) were isolated by the same method for Treg¹, except that they were from pooled DLNs from mice receiving the optimal regimen of OVA-in-8203. The responders (5×10⁴ cells/well) were cultured in RPMI 1640/10% FCS in multiple U-bottom wells with the stimulators (2×10⁵ cells/well) and the OVA323-339 peptide Antigen (0.1 μg/ml) for 3 days in the presence of Treg1 (5×10⁴ cells/well), Treg-(5×10⁴ cells/well), or neither. The cultured cells were analyzed by flow cytometry, gating on the responders (CD4+KJ1-26+Foxp3-eGFP-). Proliferation of the responders was measured by the count in percent for eFluor® 67010 Teff cells (“% eFluor® 67010 Teff”), calculated as % of total eFluor® 670⁺ Teff cells.

Analysis of Antigen-Specific Immune Suppression. The model was given the optimal regimen of OVA-in-8203 or a similar regimen of OVA-in-PBS (control). Four months later, both groups were challenged by an injection (s.c.) of 100 μg of OVA/100 μg of HEL in FCA, followed by a boost injection (s.c.) three weeks later of the same dose of antigens in FIA. KJ1-26⁺ Treg and Teff cells in the spleen were counted immediately before the start of the challenge, and five days after the end of the challenge. Delayed-type hypersensitivity (“DTH”) to HEL was analyzed three weeks after the end of the challenge by injecting the left footpad with 20 μl of PBS (control) and the right footpad with 20 μl of PBS containing 20 μg of heated HEL, which was prepared by heating HEL (1 mg/ml in PBS) at 99° C. for 20 minutes (in a PCR thermal cycler). Footpad swelling was measured at 24 hours with a Mitutoyo digital micrometer. Swelling=thickness of the right footpad-that of the left footpad. DTH to OVA was similarly analyzed after another three weeks with OVA at 20 μg/footpad. KJ1-26⁺ Teff cells in the DLN were counted five days after the OVA DTH test.

Statistics. Unpaired t test was used for comparisons between two independent groups. When multiple t tests were performed in parallel, the resulting p-values were adjusted by Bonferroni correction. One-way ANOVA was used for comparisons involving three or more independent groups, with post hoc Tukey's honest significant difference test for multiple-to-multiple comparisons, or with Bonferroni correction or Holm's method for multiple-to-one comparisons. Statistical significance was set at p≤0.05. The GraphPad Prism software was used for curve fitting.

Higher GRB: GRa ratio attributes to lower sensitivity to dexamethasone. To understand why dexamethasone has limited adjuvanticity, the GR isoform hypothesis was tested. It has been shown that Treg cells, B-1 cells, and MMos all have a lower sensitivity to dexamethasone than their respective immunogenic counterparts, i.e., the Teff cells, B-2 cells, and DCs (Chen et al. (2004) Eur. J. Immunol. 34:859-869; Chen et al. (2006) Eur. J. Immunol. 36:2139-2149; Chen et al. (2014) J. Immunol. 193:35-39; Zheng et al. (2013) Eur. J. Immunol. 43:219-227). It was therefore determined whether a similar pattern could be seen in their GR isoform ratio. This was indeed the case: Treg cells, B-1 cells, and MMos all had 3-6-fold higher GR-β/GR-α ratios in mRNA than their counterparts. In B-1 cells, knockdown of GR-α and GR-β led to decrease and increase in the cells' sensitivity to dexamethasone, respectively. Of note, the same study could not be performed in the other cell types because they could not survive the knockdown procedure. Collectively, these results support the GR isoform hypothesis. It was therefore understood that dexamethasone acts essentially as a differential inhibitor for the T, B, and antigen presenting cell types. As a result, it does not stimulate any of the cell types actively.

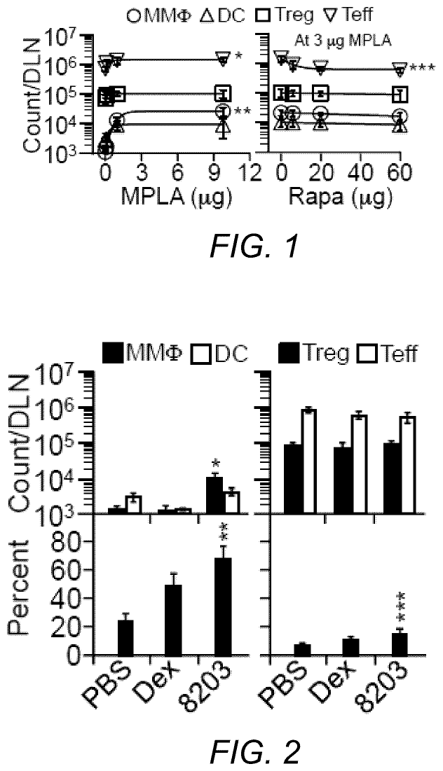

Composition 8203 potentiates draining lymph node (DLN) for Treg response. Because no other biased immunosuppressants have been shown to have the required stimulatory capacity (Northrup et al. (2017). Mol. Pharm. 14:66-80), it was posited that dexamethasone would be difficult to replace. Therefore, a biased immune stimulator was sought to complement dexamethasone in order to improve dexamethasone's adjuvanticity. From many candidates (cytokines, costimulators, proinflammatory mediators, and small molecule agonists), an unexpected combination was found, namely, monophosphoryl lipid A (“MPLA”), a lipopolysaccharide-derived conventional adjuvant that activates TLR4 (Casella & Mitchell (2008) Cell Mol. Life Sci. 65:3231-3240); and rapamycin (sirolimus), a macrolide immunosuppressant that inhibits mTOR (Abraham & Wiederrecht (1996) Annu. Rev. Immunol. 14:483-510). This pair stood out largely because of their unique interaction that attracted MMos preferentially. When MPLA was injected alone into a footpad of a BALB/c mouse, it induced an influx of MMφs and Teff cells preferentially over that of DCs and Treg cells, respectively, to the draining lymph node (“DLN,” popliteal) (FIG. 1). However, if MPLA was co-injected with rapamycin, the Teff cell influx was blocked, resulting in more selective accumulation of MMφs (FIG. 1). In BALB/c mice, the combination of 3 μg MPLA and 20 μg rapamycin was sufficient to produce the MMφ-accumulating effect (FIG. 1).

MMos are precursor cells that differentiate into Treg cell-stimulating, IL-10+tolerogenic antigen presenting cells called dexamethasone-enriched macrophages (“Dex-Mos”) (Zheng et al. (2013) Eur. J. Immunol. 43:219-227). The phenotype and function of both MMos and Dex-Mos have been described in detail (Zheng et al. (2013) Eur. J. Immunol. 43:219-227). It was therefore determined whether the above-referenced dose of 3 μg MPLA and 20 μg rapamycin may work well with dexamethasone in producing a large number of Dex-Mos. This new composition was referred to as “8203” (8 μg dexamethasone: 20 μg rapamycin: 3 μg MPLA). When injected into a footpad of a BALB/c mouse, 8203 increased the abundance of MMφs in the DLN among total antigen presenting cells by increasing the absolute number of MMos preferentially over that of DCs (FIG. 2). It also increased the abundance of Treg cells in the DLN among total CD4⁺ T cells (albeit less drastically) by increasing the absolute number of Treg cells and decreasing that of Teff cells (FIG. 2). Lymphoid organs not connected directly from the injection site, such as the contralateral brachial and axillary lymph nodes (“NLNs”) and the spleen, were less affected. In comparison, injection of dexamethasone alone was less effective compared to 8203 because dexamethasone did not increase the absolute number of MMos or Treg cells in the DLN (FIG. 2). It was therefore concluded that 8203 is a stronger potentiating agent than dexamethasone.

It was possible that the effect of 8203 is peculiar to the BALB/c strain. Thus, 8203 was tested in C57BL/6 mice as well. However, 8203 showed a similar potentiating effect in the C57BL/6 mice as was observed in BALB/c mouse.

The respective role of each component within 8203 was also determined. MPLA was found to attract MMS (FIG. 3). Although dexamethasone could enrich MMos relatively by killing DCs preferentially (FIG. 2; Zheng et al. (2013) Eur. J. Immunol. 43:219-227), this effect was masked by the much stronger effect of MPLA on the MM influx (FIG. 3). Rather, dexamethasone converted MMØs into Dex-Mos (Zheng et al. (2013) Eur. J. Immunol. 43:219-227). Rapamycin was found to block the influx of Teff cells (FIG. 3).

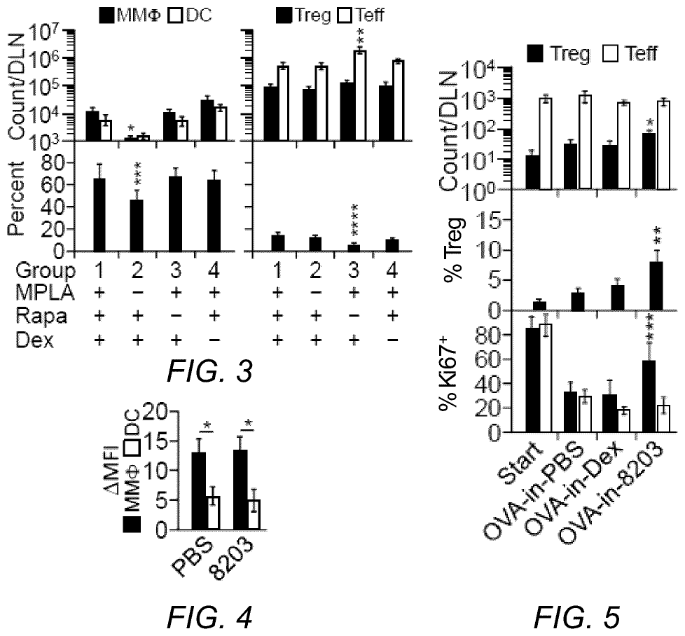

Kinetically, the effect of 8203 on MMos after a single injection peaked in 2 days in the DLN and was gone after 4 days. At the peak time, the MMos picked up and proteolytically processed DQ ovalbumin (“DQ OVA”), a model whole-protein antigen, in the DLN as actively as those in mock (PBS)-injected mice (FIG. 4).

In summary, these results show that 8203 has a stronger potentiating effect on the Treg response than does dexamethasone. This effect is local and transient and does not interfere with antigen uptake or processing. The latter property further indicates that 8203 is compatible with whole-protein antigens, the preferred form of immunogen for overcoming the HLA diversity in humans.

Model for testing tolerogenic adjuvants. To determine whether 8203's potentiating effect helps stimulate a Treg response to immunization, a mouse model was developed. BALB/c Foxp3-eGFP mice were transfused with a small number of hen ovalbumin (“OVA”)-specific TCR-transgenic CD4⁺ T cells bearing the KJ1-26+marker (˜4,000 cells from DO11.10 Foxp3-eGFP mice). By a previous estimate, ˜6% of the donor T cells (240 cells) entered the lymph nodes in the recipients (Quiel et al. (2011) Proc. Natl. Acad. Sci. USA 108:3312-3317). This number was small enough to be physiological (Moon et al. (2007) Immunity 27:203-213), yet big enough for tracking the cells.

As a potential tolerogenic adjuvant, 8203 must be able to work against already active Teff cells. To test that, the donor T cells (KJ1-26+) in the recipients were activated by injecting 100 μg of OVA into a footpad. While having a far limited effect in NLNs and the spleen, the injection expanded KJ1-26⁺ Teff cells >100-fold in the DLN five days after transfusion. Few KJ1-26⁺ Teff or Treg cells were found in other sites such as blood and bone marrow; therefore, these sites were not further studied. In the DLN, nearly all the KJ1-26⁺ Teff cells (Foxp3-) were activated by day 5, with ˜94% and ˜89% of the cells expressing the memory-stage marker CD44^{hi} and proliferation marker Ki67, respectively. In contrast, most host Teff cells (KJ1-26-Foxp3-) remained inactive, as assessed by the same markers. These results indicated that the recipients were ready to be used on day 5 as “the model.”

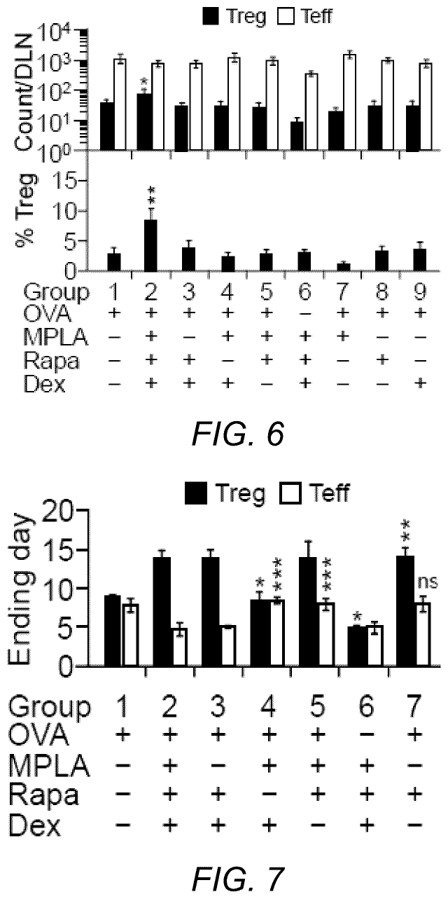

Composition 8203 is a complete tolerogenic adjuvant for Treg cells. Because in the model KJ1-26⁺ Teff cells were activated in the DLN, an injection at the primed footpad was the most direct route to the cells. Indeed, a single footpad injection of a mixed solution containing 8203 and OVA (“OVA-in-8203”) was sufficient to raise the absolute count of KJ1-26⁺ Treg cells in the DLN beyond that by an injection of the antigen alone (“OVA-in-PBS”) (FIG. 5). Such effect was not seen if dexamethasone was used in place of 8203 (“OVA-in-Dex”) (FIG. 5). Furthermore, the OVA-in-8203-expanded Treg cells also contained a larger fraction of proliferating cells identified by the proliferation marker Ki67 (FIG. 5). Thus, 8203 augmented absolute expansion of the Treg cells by increasing their proliferation. It was also noted that 8203 did so even when KJ1-26⁺ Treg (and Teff) cells underwent spontaneous reduction in proliferation (FIG. 5, “Start” versus “OVA-in-PBS”), which further revealed the stimulating effect of this adjuvant on Treg cell proliferation. This effect was OVA-specific because host Treg cells, which were mostly non-OVA-specific, were not significantly expanded and remained mostly Ki67-. In addition, this effect was OVA-dependent (FIG. 6, group 6 versus group 2), which confirmed that 8203 acted only as an adjuvant. Thus, by all accounts 8203 acted as a “true” Treg-stimulating adjuvant.

Within 8203, all three components were required for stimulating the absolute expansion of KJ1-26⁺ Treg cells (FIG. 6). All three were also required for the best relative expansion of the Treg cells within total KJ1-26⁺ T cells (FIG. 6). In aggregate, these results define 8203 as the minimum composition for a true adjuvant for Treg cells. As such, 8203 may be referred to as a “complete tolerogenic adjuvant” to distinguish it from incomplete adjuvants such as dexamethasone.

Composition 8203 augments absolute expansion of Treg cells via MMφs. The dependence of 8203 on MPLA (FIG. 6) presumably reflects the involvement of MMos. To confirm that, the number of MMos in the DLN was reduced. Intravenous injection of clodronate liposome is known to prevent influx of monocytes or macrophages into lymph nodes by selectively depleting the cells from the blood circulation (Fink et al. (2009) Eur. J. Immunol. 39:2809-2821). Thus, the experiment depicted in FIG. 5 was repeated while giving the model clodronate liposome. The results of this analysis indicated that the clodronate liposome treatment reduced the influx of MMos in the DLN by ˜73% and kept the MMφ count low during the period of Treg cell expansion (days 6-8). As a result, the adjuvant effect of 8203 in the OVA-in-8203 injected (compared to the OVA-in-PBS injected) mice was completely abolished. Thus, MMφs are an essential cell mediator for the effect of 8203.

Composition 8203 promotes systemic dissemination of expanded Treg cells. Composition 8203 also outperformed dexamethasone in augmenting the absolute KJ1-26⁺ Treg cell expansion in distant lymphoid organs (not directly draining the injection site) such as the NLNs and the spleen. But it did so mainly by dissemination because the KJ1-26⁺ Treg cells in the NLNs and spleen, unlike their counterpart in the DLN, did not have a large fraction of proliferating (i.e., Ki67+) cells. Moreover, the gain of KJ1-26⁺ Treg (but not Teff) counts there could be blocked with FTY720, an inhibitor for T cell egress from lymph nodes Cyster & Schwab (2012) Annu. Rev. Immunol. 30:69-94). In fact, the gain in KJ1-26⁺ Treg counts in the distant organs correlated with a loss of the same cells in the DLN. It was estimated that ˜55% and ˜71% of the KJ1-26⁺ Treg cells in NLNs and the spleen, respectively, came from other lymphoid organs, whereas <4% of the KJ1-26⁺ Teff cells came that way. The lack of KJ1-26⁺ Teff cell ingress in these organs correlated with the lack of KJ1-26⁺ Teff cell expansion in the DLN (FIG. 5). The latter might cause the former by reducing the number of cells available for egress. Collectively, these results indicate that the cells' systemic dissemination depends on their local expansion; and that 8203 promotes the systemic dissemination of antigen-specific Treg cells by promoting their expansion in the DLN.

Composition 8203 helps prolong Treg cell expansion. In the model, measuring the effect of a single injection of OVA-in-8203 after three days was adequate because by a kinetics study, the Treg cell expansion in the DLN peaked in three days. Led by that finding, it was further found that repeated injections of OVA-in-8203 once every 3 days expanded KJ1-26⁺ Treg cells progressively over a 9-day period, causing between 3-fold (in the spleen) to 13-fold (in the DLN) increases in the absolute cell number. In contrast, KJ1-26⁺ Teff cells declined progressively during the same period. The combination of the two opposite events resulted in a marked shift of cell balance, with the KJ1-26⁺ Treg cells representing between ˜34% (in the DLN) to ˜60% (in the spleen) of the total KJ1-26⁺ T cells in the end. This was not the case if OVA was injected without 8203 (i.e., OVA-in-PBS), which resulted in a shortened Treg cell expansion and a slightly prolonged Teff cell expansion. Focusing on these processes in the DLN, it was deteremined that the combination of rapamycin and OVA was both necessary and sufficient for prolonging the Treg cell expansion, and this action was Treg cell-selective (FIG. 7). On the other hand, the combination of rapamycin and dexamethasone was necessary for shortening the Teff cell expansion (FIG. 7), and this action was Teff cell-selective (FIG. 7). MPLA was not required here for either effect (FIG. 7). These results indicate that in mice, the optimal regimen of 8203-adjuvanted immunization is one subcutaneous injection every three days for a total of three injections.

Composition 8203 helps expand nTreg cells. To identify the type of Treg cells that 8203 helps expand, the cells' origin was traced. These cells originated from donor CD4+Foxp3⁺ Treg cells (“the source Treg cells”) because non-recipient mice or mice that received only donor CD4+Foxp3-Teff cells could not produce the Treg cells in question despite being given the optimal regimen of OVA-in-8203. The source Treg cells were nTreg cells because they existed naturally in donor mice, constituting ˜3.8% of the CD4⁺ T cells in blood at age of 8 weeks. They also expressed a set of markers typical of nTreg cells. In comparison, the Treg cells in question expressed the same set of markers at the same relative levels, with the exception of PD1, which was at a lowered level. Collectively, these findings indicate that 8203 helps expand nTreg cells.

It was possible that 8203 helped expand nTreg cells without preserving their function. Because these cells were too scarce to isolate, the cells could not be directly studied. Hence, the source Treg cells were studied as a surrogate. To that end, donor mice were given the optimal regimen of OVA-in-8203. The source Treg cells responded to the regimen by expanding their number ˜14-fold in the DLN while upregulating the TNFR2⁺ Treg activation marker (Chen & Oppenheim (2011) Immunology 133:426-433; Chen et al. (2008) J. Immunol. 180:6467-6471) in nearly half of their population. Consistent with what was seen in the model, this response was also Treg cell-biased because Teff cells in the same DLN expanded only ˜6-fold and remained mostly TNFR2⁻. Taking advantage of their abundance, the expanded source Treg cells were analyzed for their suppressive activity using an in vitro suppression assay. They were equivalent to naïve source Treg cells from untreated donor mice. Hence, nTreg cells remain functional after being expanded by 8203-adjuvanted immunization.

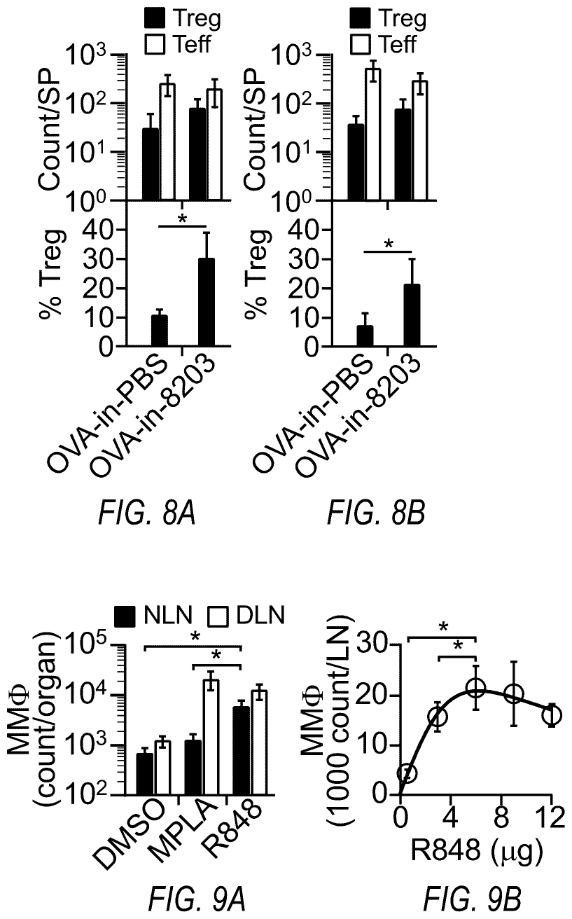

Composition 8203-adjuvanted immunization induces Antigen-specific immune suppression. To determine whether 8203-adjuvanted immunization induces antigen-specific immunosuppression, the model was given the optimal regimen of OVA-in-8203 and then challenged with OVA in the most stringent way: an injection of OVA emulsified in Freund's complete adjuvant (“FCA”), followed by a boost injection of OVA in Freund's incomplete adjuvant (“FIA”). It was reasoned that if the regimen induced OVA-specific immunosuppression, the mice would respond poorly to the challenge. The results showed that the challenge failed to lower the level of KJ1-26⁺ Treg cells in the OVA-in-8203 immunized mice to that in OVA-in-PBS immunized control mice (FIG. 8A versus FIG. 8B). The former mice also developed a weaker delayed-type sensitivity (“DTH”) to OVA than did the latter, which correlated with fewer KJ1-26⁺ Teff cells in the DLN. However, the former mice developed a normal DTH to hen egg lysozyme (“HEL”), a control antigen co-injected with OVA during the challenge. In aggregate, these results support the notion that in mice, 8203-adjuvanted immunization induces antigen-specific immunosuppression.

### EXAMPLE 2: An Antigen-Less Pro-Vaccine for Treating Autoimmunity

Mice. Use of mice was approved by the Biologic Resource Committee of University of Illinois College of Medicine at Rockford. C57BL/6, C57BL/6 Apoe^{-/-}(JAX stock #002052; Piedrahita et al. (1992) Proc. Natl. Acad. Sci. USA 89:4471-4475), C57BL/6 Foxp3-eGFP (JAX stock #006772; Lin et al. (2007) Nat. Immunol. 8:359-368), and C57BL/6 Ki67-tagRFP (JAX stock #029802; Basak et al. (2014) EMBO J. 33:2057-2068) mice were originally from The Jackson Laboratory. C57BL/6 Apoe^{-/-}Foxp3-eGFP Ki67-tagRFP (named “AFK”) and C57BL/6 Foxp3-eGFP Ki67-TagRFP (named “FK”) mice were generated by crossing the relevant strains. Other strains were as described in Example 1. AFK mice were fed a western type diet (Research Diets) starting at 10 weeks of age. They were maintained at 67° C. with extra paper bedding to prevent spontaneous skin lesions. All other strains were fed a normal chow diet. Female and male mice were used in equal numbers in all the experiments involving animals; data were pooled from both sexes because no significant sex difference was observed with the group sizes used in these experiments. The word “mice” will be used to indicate the inclusion of both sexes.

Reagents. Most reagents used were as described in Example 1. R848 (resiquimod) was from Tocris. Murine IL-2 was from PeproTech. Cell culture and reagents plates were from ThermoFisher. I-Ab-restricted epitope peptides INP (influenza nucleoprotein311-325; Crowe et al. (2006) Vaccine 24:457-467), TRP1 (tyrosinase-related protein-1113-127; Kumai et al. (2017) Cancer Immunol. Res. 5:72-83), MOG (myelin oligodendrocyte glycoprotein35-55; Hjelmstrom et al. (1998) J. Immunol. 161:4480-4483), OVA (ovalbumin323-339; Robertson et al. (2000) J. Immunol. 164:4706-4712), P210m (mouse counterpart of the human apoB-100-derived peptide 210; Wigren et al. (2011) J. Intern. Med. 269:546-556), HMW4 (HMGB198-112; Pan et al. (2016) Atherosclerosis 251:31-38), and HP1 (HSP60292-308; Chen et al. (2014) J. Immunol. 193:35-39) were synthesized by Biomatik.

Preparation and Injection of Composition 8206. Composition 8206 was prepared in the same steps as described for 8203 (Example 1), except that R848 was used in place of MPLA. It was injected subcutaneously at the back flank in 50 μl of PBS. PBS containing DMSO (as “PBS”), which was the initial solvent for rapamycin and R848, was used as a control.

Ki67-TagRFP-Based In Vitro Proliferation Assay for T Cells (“Ki67 Assay”). Total cells were isolated from lymphoid organs, washed twice in RPMI 1640/10% FCS, and counted by flow cytometry as described in Example 1. A portion of the isolated cells was cultured at 1×10⁶ cells/well in a 96-well U-bottom plate. The wells were filled with 200 μl of RPMI 1640/10% FCS supplemented with 2-mercaptoethanol (50 μM), murine IL-2 (20 ng/ml), MPLA (100 ng/ml), sodium pyruvate (1 mM), and a peptide antigen (3 μg/ml) or DMSO (as a solvent control for the antigen). After 5 days, the cells were analyzed by flow cytometry with a BD FACS Melody. Proliferating Treg and Teff cells were identified as CD4+Foxp-eGFP+Ki67-TagRFP+CD62L1o and CD4+Foxp-eGFP Ki 67-TagRFP+CD62L10 populations, respectively; the cells were counted by the method of Schlenke ((1998) Cytometry 33:310-317). Of note, CD62Lle was used here as a marker for TCR triggering (Chao et al. (1997) J. Immunol. 159:1686-1694). The count of antigen-reactive Treg (or Teff) cells =the count of proliferating Treg (or Teff) cells in the presence of a peptide antigen—that in the presence of DMSO. The difference was expressed as count/organ by multiplying the total number of cells in an organ divided by the portion used in the assay. A value of 0 was given to any negative difference to indicate that antigen-reactive cells were undetected.

Analyses of Atherosclerosis. Cytokines in blood plasma of 12 hour-fasted mice were measured with the BD Cytokine Bead Array for mouse Th1/Th2/Th17 kit (BD Biosciences). Methods for measuring triglycerides, HDL, and LDL/VLDL in plasma and quantifying atherosclerotic lesions at the aortic root were described (Pan et al. (2016) Atherosclerosis 251:31-38). Aorta (from the heart to the diaphragm) was weighted and digested to form a single cell suspension by the method of McCarthy et al. ((2013) FASEB J. 27:499-510), followed by flow cytometry with a FACS Melody. Proliferating Treg cells were identified as a CD4+CD45+Foxp-eGFP+Ki67-TagRFP+ population and counted by the method of Schlenke ((1998) Cytometry 33:310-317). Mice were sensitized to ovalbumin by priming (subcutaneously) and boosting (intraperitoneally) with 50 μg ovalbumin in AddaS03 (Invivogen). Delayed-type sensitivity to OVA was determined as previously described (Kang et al. (2008) J. Immunol. 180:5172-6).

Statistics. The statistical and curve-fitting methods were described in Example 1.

Results. An antigen-less pro-vaccine must be able to reach over-presented auto-antigens anywhere in the body (i.e., act systemically). Although two of 8203's components, namely dexamethasone and rapamycin, act systemically, the third component, MPLA, acts locally. As a result, the adjuvant effect of 8203 is at DLNs near the site of administration. Therefore, MPLA was replaced with a systemically-acting TLR7/8 agonist R848 (resiquimod) (Tomai et al. (2007) Expert Rev. Vaccines 6:835-847). Like MPLA, R848 attracted MMos to the DLNs (FIG. 9). Indeed, R848 could attract MMos to non-draining lymph nodes (NLNs) as well, whereas MPLA could not (FIG. 9). This was likely attributed partly to the much smaller molecular weight of R848 (314 Da) in comparison with that of MPLA (1763 Da). The optimal dose of R848 for this systemic effect was about 6 μg when combined with 8 μg of dexamethasone and 20 μg of rapamycin. Accordingly, MPLA was replaced with 6 μg of R848 to create the composition referred to as “8206.”

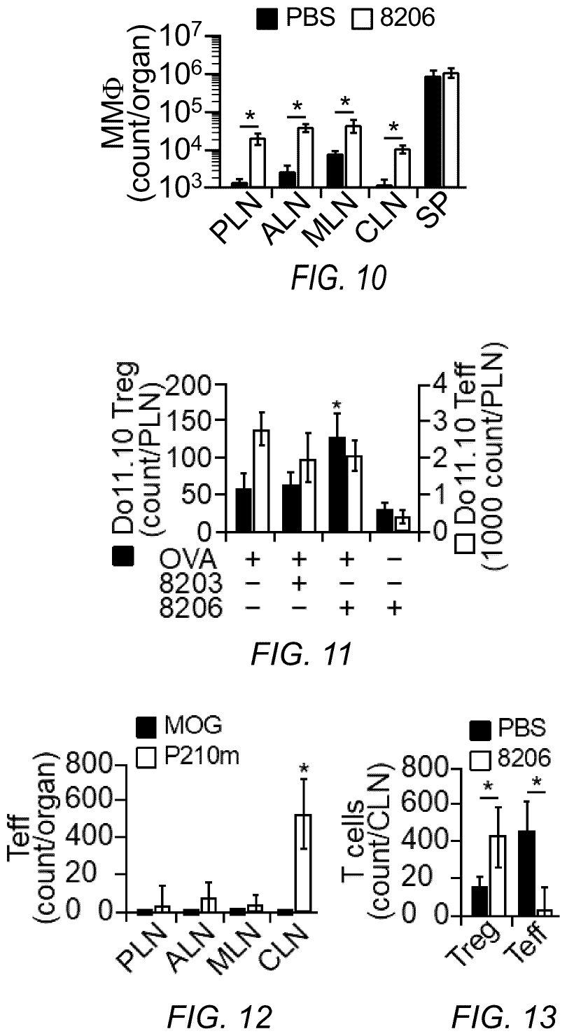

With its three components all acting systemically, composition 8206 showed potent systemic adjuvant activity. A single subcutaneous injection of composition 8206 at the back flank of a mouse caused an influx of MMos in NLNs throughout its body, such as the popliteal lymph nodes (PLN) in the knees, axillary lymph nodes (ALN) in the shoulders, caudal mediastinal lymph node (CLN) in the chest cavity, and mesenteric lymph nodes (MLN) in the abdominal cavity (FIG. 10). The spleen was minimally affected (FIG. 10). Moreover, in BALB/c mice adoptively transferred with DO11.10 TCR-transgenic T cells (Example 1), the injection of composition 8206 at the back flank stimulated the expansion of OVA-reactive Treg cells in a PLN, where OVA was over-presented (FIG. 11). Importantly, the effect of composition 8206 was antigen (OVA)-dependent (FIG. 11). It was therefore concluded that composition 8206 was a systemically-acting complete tolerogenic adjuvant.

It was subsequently determined whether composition 8206 was an active vaccine with endogenous auto-antigens. In this analysis, low-density lipoprotein (LDL), one of the most abundant and pathogenically significant auto-antigens in the body, was used. LDL promotes atherosclerosis in a pathogenic process that involves T cell-mediated autoimmunity against apolipoprotein B-100 (ApoB) (Marchini et al. (2021) Hamostaseologie 41:447-457), a large glycoprotein on the surface of LDL. This autoimmunity can be modeled in the Apoe^{-/-}mice where a high-fat and high-cholesterol western-type diet (WTD) is used to rapidly raise LDL and its ApoB and accelerate atherosclerosis. It was therefore determined whether composition 8206 would work with endogenous ApoB in this model.

To that end, WTD was fed to a sub-strain of the Apoe^{-/-}mice, namely the AFK mice (described herein). Lymph nodes where ApoB was over-presented were analyzed as the sites of interest. By the definition of over-presentation given earlier, these lymph nodes should contain more ApoB-reactive Teff cells than do other lymph nodes. Here, focus was placed on P210m, a mouse counterpart of the well-studied human ApoB epitope P210 (Chyu et al. (2017) Ther. Adv. Vaccines 5:39-47). It was found that among the four lymph nodes (LNs) studied in FIG. 10, the CLN contained the most P210m-reactive Teff cells (FIG. 12), and it was also noticeably enlarged in response to WTD. After a single subcutaneous injection of composition 8206, the mice showed a significant increase in P210m-reactive Treg cells and a significant decrease in P210m-reactive Teff cells (both in absolute numbers) inside the CLN (FIG. 13). P210m-reactive Treg cells were not detected in the other three LNs. Thus, while composition 8206 increased MMos in all LNs (as shown in FIG. 10), it caused an expansion of Treg cells only in the LN where the auto-antigen was over-presented. Kinetically, the Treg cell expansion in the CLN peaked in three days. In five days, P210m-reactive Treg cells accumulated in the spleen; such accumulation was likely due to systemic dissemination of Treg cells from the CLN and possibly other LNs with active anti-ApoB autoimmunity because it could be inhibited with the egress blocker FTY720. Similar systemic dissemination of Treg cells after expansion of Treg cells in a LN was similarly noted with the parental composition 8203 (Example 1). In aggregate, these results showed that composition 8206 formed a vaccine with endogenous ApoB and did so in a site-specific manner.

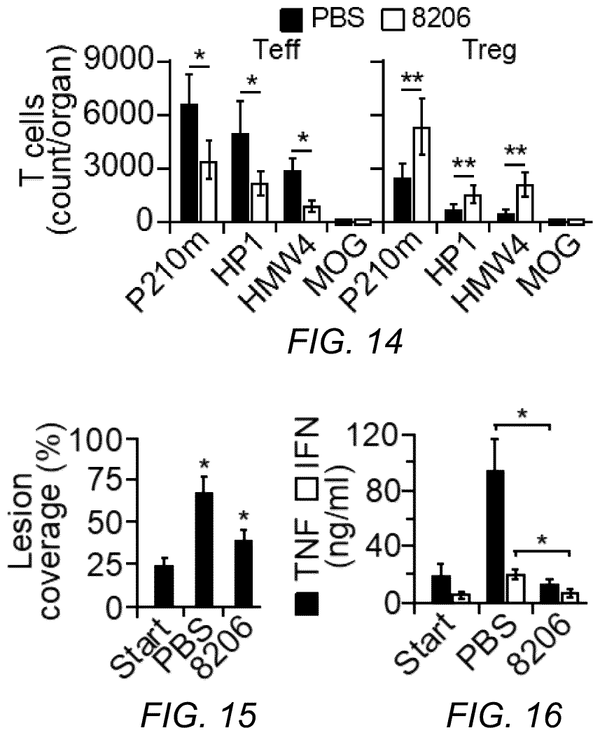

There are at least two other auto-antigens known to be over-presented in this model, HSP60 (Wick et al. (2014) Nat. Rev. Cardiol. 11:516-529) and HMGB1 (Pan et al. (2016) Atherosclerosis 251:31-38). The serum level of ApoB is on the order of 100 mg/dL (Wagner et al. (2019) J. Lipid Res. 60:900-908), whereas those of HSP60 and HMGB1 are on the order of 1 μg/dL (Pan et al. (2016) Atherosclerosis 251:31-38; Zhang et al. (2015) Int. J. Environ. Res. Publich Health 12:5743-5757). TO determine whether composition 8206 would also work with HSP60 and HMGB1, accumulation of HSP60- and HMGB1-reactive Treg cells in the spleen was investigated. Here, HP1 and HMW4, which are specific epitopes of HSP60 and HMGB1, respectively, were used (Pan et al. (2016) Atherosclerosis 251:31-38; Chen et al. (2014) J. Immunol. 193:35-39). It was observed that five days after a single subcutaneous injection of composition 8206, the spleen gained HP1- and HMW4-reactive Treg cells alongside with P210m-reactive Treg cells, in the order of P210m >HMW4 and HP1 (FIG. 14). It was clear that although these over-presented auto-antigens differ in protein sequence and abundance, composition 8206 formed vaccines with each of them.

To determine whether composition 8206 worked with all the auto-antigens because they were over-presented, four non-over-presented antigens were sampled in AFK mice. Two of them were allo-antigens OVA and INP, which should be absent in the mice because the latter were not in contact with these foreign antigens; the other two were endogenous auto-antigens TRP1 and MOG, which should be present but not over-presented because the mice did not have autoimmune depigmentation (Kumai et al. (2017) Cancer Immunol. Res. 5:72-83) or autoimmune encephalomyelitis (Hjelmstrom, et al. (1998) J. Immunol. 161:4480-4483). Five days after the same injection of composition 8206, AFK mice showed gain of P210m-reative Treg cells in the spleen as before, but there was no similar gain of OVA-, INP-, TRP1-, or MOG-reactive Treg cells.

For further analysis, the same injection of composition 8206 was given to a non-atherosclerosis-prone control strain of mice, namely the FK mice (described herein), where ApoB, HSP60, and HMGB1 should not be over-presented. In contrast to AFK mice, the FK mice had minimal P210m-, HP1-, or HMW4-reactive Treg cells in the spleen and showed no significant gain after receiving composition 8206. Altogether, the seven cases where antigens were not over-presented, combined with the three cases where antigens were over-presented, showed a 99.9% probability (i.e., P=1-0. 510) that composition 8206 works only with, and thereby targets, over-presented antigens.

With its ability to target over-presented antigens, it was determined whether composition 8206 could be used for treating an antigen-associated disease. For this analysis, 10-week-old AFK mice were fed WTD for six weeks to induce atherosclerosis (FIG. 15). This was followed by four additional months of WTD to allow the disease to progress, during which period the mice were treated with once-per-month injections of composition 8206 on the first day of each month. The results, obtained on the last day of the last month, showed that the disease progressed under the pressure of WTD, but composition 8206 slowed the rate of progression to about ⅓ of that in mock (PBS)-treated mice (FIG. 15). The slowed rate correlated with a 5-fold increase in proliferating Treg cells in the aorta, which appeared to be attributed to both a 2.1-fold increase in composition 8206-treated group and a 2.4-fold decrease in the mock-treated group. It also correlated with significant decreases in TNF-α and IFN-γ in blood (FIG. 16), which may be secondary to regulation by the Treg cells. The treatment did not alter body weight or lipid profile, nor did it cause general immunosuppression because the treated mice could be sensitized normally to OVA by immunization. In aggregate, these results indicate that composition 8206 inhibited the progression of atherosclerosis by raising Treg cells at the site of disease.

What is claimed is:

1. A complete tolerogenic adjuvant composition comprising an effective amount of dexamethasone, an effective amount of rapamycin, and an effective amount of a Toll-like receptor agonist that increases influx of monocyte-derived macrophages into lymph nodes.

2. The complete tolerogenic adjuvant composition of claim 1, wherein the Toll-like receptor agonist is a TLR7 agonist, TLR8 agonist, and/or TLR4 agonist.

3. The complete tolerogenic adjuvant composition of claim 1, wherein the Toll-like receptor agonist comprises monophosphoryl lipid A or resiquimod.

4. The complete tolerogenic adjuvant composition of claim 1, wherein the dexamethasone, rapamycin, and Toll-like receptor agonist are in a mass ratio of about 6 to about 10 dexamethasone: about 15 to about 25 rapamycin: about 1 to about 8 Toll-like receptor agonist.

5. The complete tolerogenic adjuvant composition of claim 1, wherein the dexamethasone, rapamycin, and Toll-like receptor agonist are in a mass ratio of about 8:20:3 or about 8:20:6.

6. A vaccine comprising the complete tolerogenic adjuvant composition of claim 1.

7. The vaccine of claim 6, further comprising an antigen associated with a disease or condition.

8. The vaccine of claim 7, wherein the antigen is an autoimmune antigen, allergen, and/or alloantigen.

9. A method of inducing regulatory T cell-mediated antigen-specific immunosuppression in a subject comprising administering to the subject an effective amount of the complete tolerogenic adjuvant composition of claim 1 thereby inducing regulatory T cell-mediated antigen-specific immunosuppression in the subject.

10. The method of claim 9, wherein the antigen is an autoantigen and the complete tolerogenic adjuvant composition is administered in the absence of the autoantigen.

11. The method of claim 9, wherein the complete tolerogenic adjuvant composition is administered in combination with the antigen.

12. A method for treating or reducing the risk of developing an autoimmune disease or condition comprising administering to a subject in need thereof an effective amount of the complete tolerogenic adjuvant composition of claim 1, thereby treating or reducing the risk of developing the autoimmune disease or condition.

13. The method of claim 12, wherein the complete tolerogenic adjuvant composition is administered in the absence of an antigen associated with the autoimmune disease or condition.

14. The method of claim 12, wherein the complete tolerogenic adjuvant composition is administered in combination with an antigen associated with the autoimmune disease or condition.

15. A method for treating or reducing the risk of developing an allergy comprising administering to a subject in need thereof an effective amount of the complete tolerogenic adjuvant composition of claim 1, thereby treating or reducing the risk of developing the allergy.

16. The method of claim 15, wherein the complete tolerogenic adjuvant composition is administered in combination with an antigen associated with the allergy.

17. A method for treating or reducing the risk of developing transplant rejection or graft versus host disease comprising administering to a subject in need thereof an effective amount of the complete tolerogenic adjuvant composition of claim 1, thereby treating or reducing the risk of developing transplant rejection or graft versus host disease.

18. The method of claim 17, wherein the complete tolerogenic adjuvant composition is administered in combination with an antigen associated with transplant rejection or graft versus host disease.
